(19) United States
(12) Patent Application Publication
(10) Pub. No.: US 2009/0105782 A1
(43) Pub. Date: Apr. 23, 2009

(54) VAGUS NERVE STIMULATION APPARATUS, AND ASSOCIATED METHODS

(75) Inventors: Marlin H. Mickle, Pittsburgh, PA (US); Steven A. Hackworth, Pittsburgh, PA (US); Michael R. Lovell, Pittsburgh, PA (US); Doreen K. Jacob, Pittsburgh, PA (US); Theodora Bennett, Pittsburgh, PA (US); Robert Cuda, Pittsburgh, PA (US); Kathy Hoelzle, Homestead, PA (US); Eric Laurenson, Glenshaw, PA (US)

Correspondence Address: ECKERT SEAMANS CHERIN & MELLOTT, 600 GRANT STREET, 44TH FLOOR, PITTSBURGH, PA, 15219, US

(73) Assignee: University of Pittsburgh-Of the Commonwealth System of Higher Education

(21) Appl. No.: 11/683,149

(22) Filed: Mar. 7, 2007

Related U.S. Application Data
(60) Provisional application No. 60/782,440, filed on Mar. 15, 2006.

(51) Int. Cl. A61N 1/378; A61N 1/36

(52) U.S. Cl. 607/45; 607/61

(57) ABSTRACT

Methods and apparatus for providing vagus nerve stimulation for the treatment of diseases such as depression and epilepsy that do not require an onboard, implanted power supply. Power may be supplied from outside of the body by near-field inductive coupling with an external power supply provided in a support article (e.g., garment) worn by the patient. Power may also be supplied by providing an antenna for harvesting ambient RF energy and converting it into DC power. In addition, the methods and apparatus provide for remote, wireless programming of the parameters that specify the nature of current pulses provided to the vagus nerve by probes implanted in the body of the patient. The preferred stimulation profile is 1-2 milliamp pulses of 250 microseconds in duration at a frequency of 20 to 30 Hz, wherein the profile is repeatedly on for 30 seconds and off for 5 minutes.

### CROSS-REFERENCE TO RELATED APPLICATION

This application claims the benefit of U.S. Provisional Application No. 60/782,440, entitled “Vagus Nerve Stimulation for Epilepsy and Related Peripheral Nerve Stimulation,” which was filed on Mar. 15, 2006, the disclosure of which is incorporated herein by reference.

### GOVERNMENT CONTRACT

This work was supported in part by a grant from the National Science Foundation under Contract No. EEC 0502035. The United States government may have certain rights in the invention described herein.

### FIELD OF THE INVENTION

The present invention relates to methods and apparatus for providing treatment for the symptoms of various diseases, such as depression and epilepsy, and in particular to improved methods and apparatus for providing vagus nerve electrical stimulation.

### BACKGROUND OF THE INVENTION

It is known that stimulation of the vagus nerve in patients can be used as a form of therapy for the treatment of depression, particularly treatment resistant patients. There are approximately 11 million such patients in the world and approximately 4 million such patients in the United States. On Jul. 15, 2005, the United States Food and Drug Administration (FDA) approved the Vagus Nerve Stimulation (VNS) Therapy System sold by Cyberonics, Inc “for the adjunctive long-term treatment of chronic or recurrent depression for patients 18 years of age or older who are experiencing a major depressive episode and have not had an adequate response to four or more adequate antidepressant treatments.” The VNS Therapy System had been previously approved for the treatment of epilepsy. VNS therapy is delivered from a small pacemaker-like generator implanted in the chest that sends preprogrammed, intermittent, mild electrical pulses through the vagus nerve in the neck to the brain. The current device, however, requires the implantation of a relatively large battery and control pack in the body of the patient with subcutaneous wires threaded through the body to implanted probes (one or more) operatively coupled to the vagus nerve in the left side of the neck. The battery and control pack and wires may, in some cases, be a source of irritation and infection, which may require antibiotics or even removal of the device. Furthermore, the current device is susceptible to a limited battery life and magnetic interference. After the lifespan of an implant's battery, another surgery is required to replace the device. Thus, it would be advantageous to be able to provide vagus nerve stimulation in a manner that eliminates the intrusive battery pack and wires, as well as the health risks commonly associated with them.

### SUMMARY OF THE INVENTION

In one embodiment, the invention provides an apparatus for providing electrical stimulation to the vagus nerve of a patient that includes one or more probes for being implanted in the body of the patient for providing current pulses to the vagus nerve, an implantable device for being implanted in the body of the patient having: (i) control circuitry electrically connected to the one or more probes and structured to generate the current pulses and provide the current pulses to the one or more probes, and (ii) power circuitry electrically connected to the control circuitry for providing a DC power signal to the control circuitry, and a power supply separate from the implantable device and external to the patient's body. The power supply provides power to the implantable device through a near-field technique, such as near-field inductive coupling, between the power supply and the power circuitry when the power circuitry is in proximity with the power supply. The power supply of the apparatus may be provided as part of an article to be worn by the patient, such as a garment. Alternatively, the power supply may be provided at a stationary location separate from the implantable device, such as in a piece of furniture.

The control circuitry of the apparatus may include a programmable processor that controls the generation of the current pulses based upon one or more pulse parameters and a wireless communications device. The apparatus in this embodiment further includes a remote programming device external to the patient's body that is structured to wirelessly transmit programming signals to the wireless communications device for adjusting the one or more pulse parameters. The one or more pulse parameters specify one or more of a frequency, an amplitude, a pulse width, an on/off state, and an application location of the current pulses, the application location being determined by the particular ones of the one or more probes to which the current pulses are provided. The power may be provided to the implantable device and the one or more pulse parameters may be adjusted simultaneously.

Similarly, the invention also provides a method of providing electrical stimulation to the vagus nerve of a patient that includes steps of implanting one or more probes into the body of the patient, wherein the one or more probes are structured to provide current pulses to the vagus nerve, implanting a device in the body of the patient that is electrically connected to the one or more probes, causing the device to generate the current pulses and provide the current pulses to the one or more probes, and providing power to the device from a location external to the body of the patient using a near-field technique such as near-field inductive coupling.

In another embodiment, the invention provides an apparatus for providing electrical stimulation to the vagus nerve of a patient that includes one or more probes for being implanted in the body of the patient for providing current pulses to the vagus nerve and an implantable device for being implanted in the body of the patient. The implantable device in this embodiment includes control circuitry electrically connected to the one or more probes that is structured to generate the current pulses and provide the current pulses to the one or more probes and power circuitry electrically connected to the control circuitry. The power circuitry has an antenna for receiving energy transmitted in space from a far-field source, such as a local radio station or another remote RF source. The power circuitry converts the received energy into a DC power signal and provides the DC power signal to the control circuitry.

As in the embodiment described above, the control circuitry of the apparatus may include a programmable processor that controls the generation of the current pulses based upon one or more pulse parameters and a wireless communications device. The apparatus in this embodiment further includes a remote programming device external to the patient's body that is structured to wirelessly transmit programming signals to the wireless communications device for adjusting the one or more pulse parameters. The energy may be received from the far field source and the one or more pulse parameters may be adjusted simultaneously.

Similarly, the invention provides a method of providing electrical stimulation to the vagus nerve of a patient that includes steps of implanting one or more probes into the body of the patient, wherein the one or more probes are structured to provide current pulses to the vagus nerve, implanting a device in the body of the patient that is electrically connected to the one or more probes, causing the device to generate the current pulses and provide the current pulses to the one or more probes, and providing power to the device by receiving energy transmitted in space from a remote far-field source external to the body of the patient and converting the received energy into a DC power signal.

It is an object of this invention to provide a method and apparatus for providing vagus nerve stimulation that does not require an onboard power supply that is implanted within the body of the patient.

It is a further object of this invention to provide a method and apparatus for providing vagus nerve stimulation that eliminates the problems associated with the subcutaneous wires that are present with prior art devices.

It is still a further object of this invention to provide a method and apparatus for providing vagus nerve stimulation that eliminates the battery life and replacement problems present with prior art devices.

It is still a further object of this invention to provide a method and apparatus for providing vagus nerve stimulation that is powered by a near-field technique, such as near-field inductive coupling.

It is still a further object of this invention to provide a method and apparatus for providing vagus nerve stimulation that is powered by a receiving energy transmitted in space from a far-field source and converting the received energy into a DC power signal.

It is still a further object of this invention to provide a method and apparatus for providing vagus nerve stimulation that allows the current pulse parameters to be readily and non-intrusively adjusted from outside of the body.

It is still a further object of this invention to provide a method of treating a disease such as depression or epilepsy.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a block diagram of a VNS device according to a first embodiment of the present invention;

FIG. 2 is a block diagram of control circuitry for driving the probes of the VNS device of FIG. 1 according to one embodiment of the invention;

FIG. 3 is a schematic illustration of the parameters used to specify the current pulses used in the present invention;

FIG. 4 is a block diagram of a remote programming device that allows an operator to set pulsing parameters for the VNS devices described herein;

FIG. 5 is a block diagram of an implantable VNS device according to an alternative embodiment of the present invention; and

FIG. 6 is a block diagram of a VNS device according to a further alternative embodiment of the present invention.

### DESCRIPTION OF THE PREFERRED EMBODIMENTS

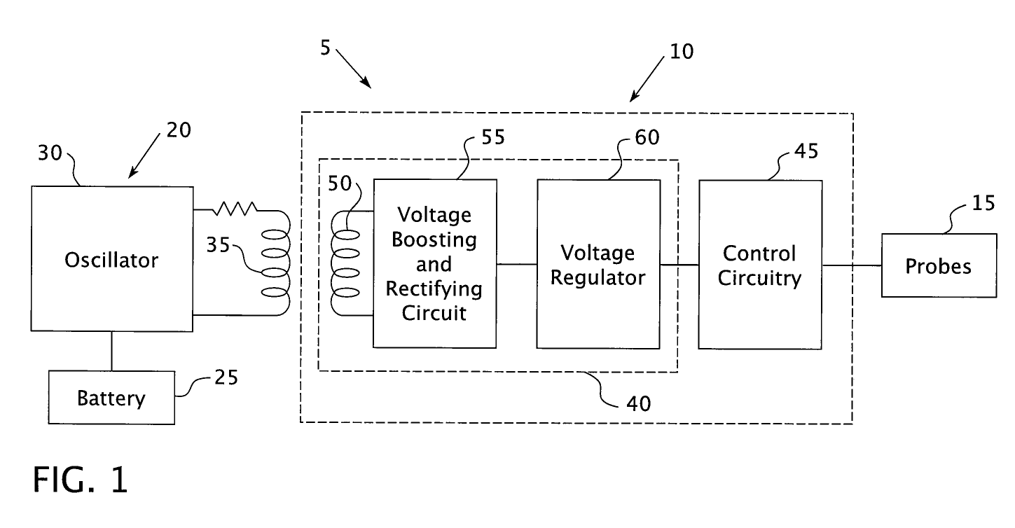

FIG. 1 is a block diagram of a vagus nerve stimulation (VNS) device 5 according to a first embodiment of the present invention for use in providing treatment to a patient, which preferably is a human, but may even include an animal. The VNS device 5 includes an implantable device 10 that is implanted in the body of the patient, preferably at or near the left side of the neck of the patient, although alternative suitable locations may also be used. In addition, because the implantable device 10 is implanted in the body, the components thereof are provided on some type of biologically compatible substrate and encased in some type of biologically compatible material, such as a substrate or housing made from an accepted medical polymer. As described in greater detail herein, the implantable device 10 controls and drives one or more probes 15 which are implanted in the neck of the patient in proximity to the vagus nerve of the patient by generating and providing to the probes 15 appropriate current pulses. The probes 15, in turn, are operatively coupled to and therefore administer the current pulses to the vagus nerve of the patient. Typically, each probe 15 is an elongated member that includes one or more electrodes along its length for actually applying the current pulses to the vagus nerve. Attachment of the probes 15 that provide the current pulses should not involve the superior cervical cardiac branch or the inferior cervical cardiac branch of the vagus nerve. The probes 15 should be placed below the area where these two branches separate from the rest of the vagus nerve.

As will be appreciated, the electronic components of the implantable device 10 require power in order to operate. The implantable device 10 does not, however, have an onboard power supply such as a battery. Instead, the embodiment of the implantable device 10 shown in FIG. 1 is remotely powered using a near-field technique, which in the embodiment shown in FIG. 1 is near-field inductive coupling. The definition of the near-field is generally accepted as a region that is in proximity to an antenna or another radiating structure where the electric and magnetic fields do not have a plane-wave characteristic but vary greatly from one point to another. Furthermore, the near-field can be subdivided into two regions which are named the reactive near field and the radiating near field. The reactive near-field is closest to the radiating antenna and contains almost all of the stored energy, whereas the radiating near-field is where the radiation field is dominant over the reactive field but does not posses plane-wave characteristics and is complicated in structure. This is in contrast to the far-field, which is generally defined as the region where the electromagnetic field has a plane-wave characteristic, i.e. it has a uniform distribution of the electric and magnetic field strength in planes transverse to the direction of propagation. As used herein, the terms near-field and far-field shall have the meaning provided above.

In the embodiment shown in FIG. 1 where near-field inductive coupling is used, the VNS device 5 includes a separate, external power supply 20 that is, in one particular embodiment, provided in support article, such as a garment, worn by the patient. The power supply 20 includes a battery 25 or some other suitable alternative energy source that is electrically connected to an adjustable oscillator 30 which generates an AC signal. A suitable example of an oscillator that may be used for the oscillator 30 is the LTC6900 precision low power oscillator sold by Linear Technology Corporation of Milpitas, Calif., which is capable of generating 50% duty cycle square waves at frequencies of between 1 KHz and 20 MHz. Other types/shapes of waveforms and/or duty cycles may also be used. The power supply also includes a primary winding 35 that is electrically connected to the oscillator 30 and receives the waveform generated thereby.

The implantable device 10 is provided with power circuitry 40 that provides a DC signal of an appropriate level for powering the control circuitry 45 provided as part of the implantable device 10. As described in greater detail herein, the control circuitry 45 controls the generation of the current pulses provided to the probes 15 (and ultimately to the patient's vagus nerve). As seen in FIG. 1, the power circuitry 40 includes a secondary winding 50, a voltage boosting and rectifying circuit 55 and a voltage regulator 60. In operation, when the AC signal is provided to the primary winding 35, a second AC signal is induced in the secondary winding 50 as a result of near-field inductive coupling with the primary winding 35.

Because of losses that occur in the inductive coupling, it is preferred to increase the voltage of the induced AC signal in order to provide a supply voltage of an appropriate level to the control circuitry 45 (as described hereinafter, the highest voltage necessary for the control circuitry 45 is typically 3 V, and the required voltage ranges from 1.5 V to 3 V, although voltages to 5 V may also be desired). In addition, because a DC signal is employed to power the control circuitry 45, the induced AC signal is also converted to DC. Thus, the induced AC signal is provided to the voltage boosting and rectifying circuit 55, which increases the voltage of and rectifies the received AC signal. In one particular embodiment, the voltage boosting and rectifying circuit 55 is a one or more stage charge pump, sometimes referred to as a “voltage multiplier.” Charge pumps are well known in the art. Basically, one stage of a charge pump increases (e.g. doubles) the amplitude of an AC input voltage and may store the increased DC voltage on an output capacitor. Successive stages of a charge pump, if present, will further increase the voltage from the previous stage. The DC signal that is output by the voltage boosting and rectifying circuit 55 is provided to a voltage regulator 60, which in turn provides a regulated DC voltage signal to the control circuitry 45. The voltage regulator 60 is primarily provided to resist spikes in the DC voltage signal provided to the control circuitry 45 and to resist DC voltage signals that may overdrive the control circuitry 45.

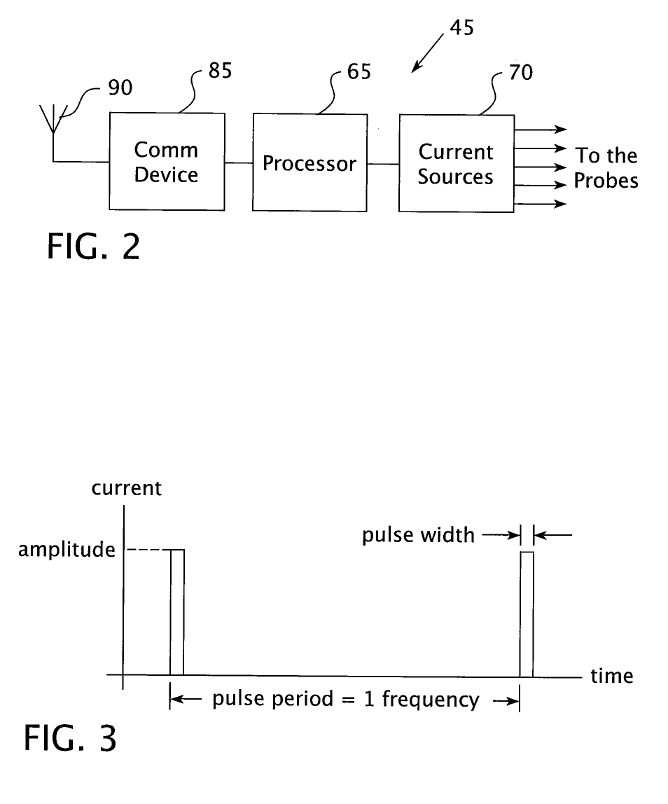

FIG. 2 is a block diagram of the control circuitry 45 for driving the probes 15 according to one embodiment of the invention. The control circuitry 45 includes a processor 65, such as a microcontroller or some other type of microprocessor. A suitable example of the processor 65 is the PIC16LF87 microcontroller sold by Microchip technology, Inc. of Chandler, Ariz. The processor 65 is programmed to output signals which cause the appropriate current pulses to be supplied to the probes 15, as well as determine to which electrode locations on the probes 15 the actual pulses are sent. As described elsewhere herein, known VNS devices exist and therefore appropriate current stimulation profiles and ranges of parameters are well understood. The nature of the current pulses provided to the vagus nerve is determined by the following five parameters: (1) frequency, (2) amplitude, (3) pulse width, (4) on/off state (i.e., whether pulses are generated and/or provided to any electrodes at all), and (5) application location (i.e., to which particular electrodes the pulses are applied). These parameters are illustrated in FIG. 3. The preferred stimulation profile is 1-2 milliamp pulses of 250 microseconds in duration at a frequency of 20 to 30 Hz, wherein the profile is repeatedly on for 30 seconds and off for 5 minutes.

In the particular embodiment of the VNS device 5 shown in FIGS. 1 and 2, the probes 15 include comprise multiple probes each having one or more electrodes for providing current pulses to any one or any combination of locations on the vagus nerve. In addition, in the particular embodiment of the VNS device 5 shown in FIGS. 1 and 2, the amplitude, frequency and pulse width of each of the current pulses that are provided to the probes 15 may be varied. It will be appreciated, however, that this embodiment is meant to be exemplary only and that more or less probes each having more or less electrodes may be employed in a device without departing from the scope of the present invention. The actual current pulses that are created and to which location or locations (i.e., which probes) they are provided is determined by parameters that, as noted above, are programmed in the processor 65. It is important in any VNS device for these parameters to be selectively adjustable, as the appropriate pulse frequency, amplitude and width must be selected and possibly later adjusted for each individual patient. Thus, the VNS device 5 of the present invention is, as described in greater detail herein, provided with a mechanism for selectively adjusting these parameters.

As stated above, the processor 65 (FIG. 2) creates and outputs signals according to the selected pulse parameters which drive current sources 70. The current sources 70 are each operatively coupled to respective probes 15 for providing the current pulses according to the selected parameters to the probes 15. The probes 15 then, in turn, provide the current pulses to the patient's vagus nerve. As noted above, in the preferred embodiment, each probe 15 is caused to output current pulses according to the following stimulation profile: 1-2 milliamp pulses of 250 microseconds in duration at a frequency of 20 to 30 Hz, wherein the profile is repeatedly on for 30 seconds and off for 5 minutes.

According to an aspect of the present invention, the implantable device 10 is adapted to preserve power when current pulsing is not required. Specifically, the processor 65 includes a watchdog timer, and the watchdog timer timeout, used as the wakeup mechanism, can be scaled down so that the processor 65 enters a sleep mode between current pulses. In addition, a low power RC oscillator external to the processor 65 may be used with the processor 65 for clocking purposes such that its internal, high speed oscillator can be turned off to further persevere power.

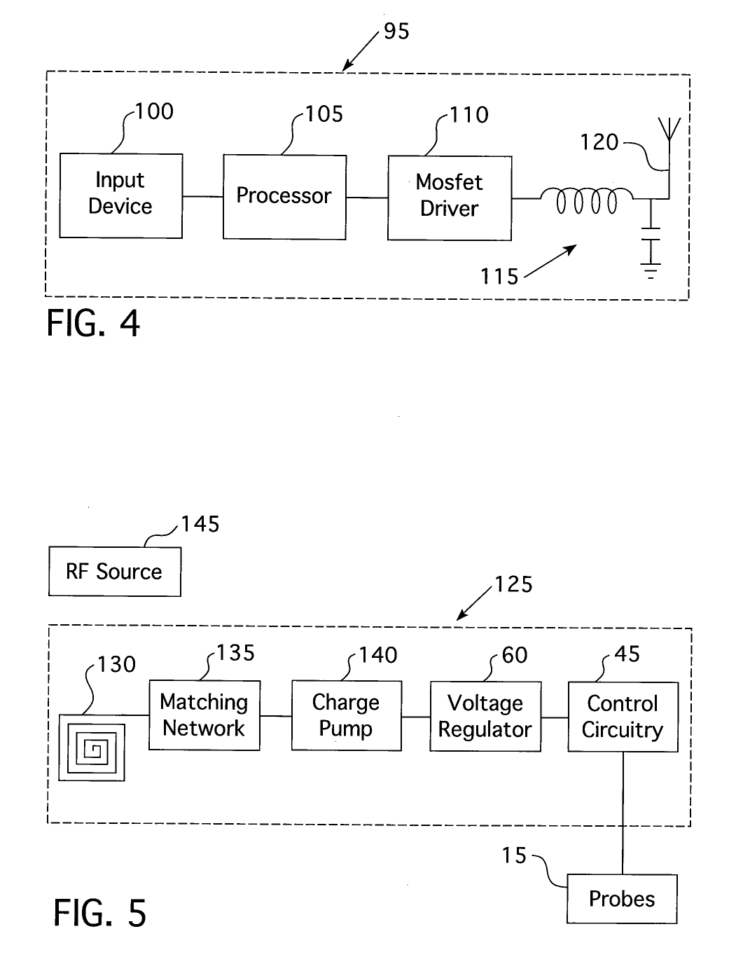

As noted above, it is preferred to be able to selectively adjust the pulsing parameters within the processor 65. Thus, according to a further aspect of the present invention, the VNS device 5 is provided with a mechanism for remotely and wirelessly programming the processor 65 so that the pulse parameters can be selectively adjusted. For this purpose, the control circuitry 45 includes a wireless communications device 85 having an antenna 90 that is in electronic communication with the processor 65 when it is necessary to perform adjustments. The wireless communications device 85 is adapted to receive programming signals sent from a remote programming device 95 shown in block diagram form in FIG. 4 and described hereinafter. The wireless communications device 85 may be any wireless receiver or transceiver that is able to communicate via any of a number of known wireless communications protocols, including, without limitation, an RF protocol such as BLUETOOTH®. A suitable device that may be used for the wireless communications device 85 is the ATA5283 low power receiver that was sold by Atmel Corporation of San Jose, Calif. That particular device uses a simple ASK protocol at a frequency of 125 KHz and stays in a standby (low power sleep) mode until it senses a 125 KHz preamble of at least 5.64 ms, after which it wakes up and outputs digital data based on the presence of the 125 KHz signal. After data transmission, a simple digital high input to the reset pin puts the device back to sleep. The antenna used in this application is a small wire wrapped around the circuitry perimeter, although other forms are possible. It should be noted that the wireless adjustment of the pulsing parameters and the powering of the device may occur simultaneously.

FIG. 4 is a block diagram of the remote programming device 95 that allows an operator to set pulsing parameters for the VNS device 5 and transmits programming signals which will cause the processor 65 to implement the selected parameters. The remote programming device 95 includes an input device 100 that enables an operator to set desired programming values. The input device 100 may be any suitable mechanism for inputting data, such as, without limitation, a keypad, a touch screen, or a series of slide switches. The input device 100 is in electronic communication with a processor 105 so that the data input by the operator can be sent thereto. The processor 105 is adapted to receive the input signals relating to the desired pulse parameters and convert them into programming signals appropriate for programming the processor 65 of the control circuitry 45. The processor 105 is preferably a microcontroller such as the PIC16LF87 microcontroller sold by Microchip technology, Inc. of Chandler, Ariz. Most suitable processors are not able to create a healthy sinusoid for transmitting the programming signals. As a result, in order to generate a signal appropriate for transmission, the processor 105 sends the programming signal pulses to a MOSFET driver 110, such as the TC4422 driver sold by Microchip corporation, provided as part of the remote programming device 95 which in turn drives an LC circuit 115 also provided as part of the remote programming device 95. The MOSFET driver 110 is powered by a separate 12 V power supply (not shown) so as to provide enough current to drive the high voltage and current oscillations in the LC circuit 115. In addition, the LC circuit 115 alone is not sufficient to send a strong signal to the control circuitry 45 (FIG. 1), but instead employs an antenna 120 to transmit the 125 KHz signal more efficiently. For this purpose, a PhidgetRFID antenna sold by Phidgets Inc, Calgary, Canada, designed for use with 125 KHz RFID systems, may be used for antenna 120. It will be appreciated that other suitable wireless transmitting devices, such as various commercially available transmitter and/or transceiver chips and antennas, may also be used without departing from the scope of the present invention.

FIG. 5 is a block diagram of an implantable VNS device 125 connected to implanted probes 15 according to an alternative embodiment of the present invention. The VNS device 125, like the VNS device 5, is adapted to be implanted in the body of the patient in proximity to the patient's vagus nerve. In addition, the VNS device 125 does not have an onboard power supply such as a battery. Instead, the VNS device 125 is powered by harvesting energy that is transmitted in space form a far-field source. As employed herein, the term “in space” means that energy or signals are being transmitted through the air or similar medium regardless of whether the transmission is within or partially within an enclosure, as contrasted with transmission of electrical energy by a hard wired or printed circuit boards. A number of methods and apparatus for harvesting energy from space and using the harvested energy to power an electronic device are described in U.S. Pat. No. 6,289,237, entitled “Apparatus for Energizing a Remote Station and Related Method,” U.S. Pat. No. 6,615,074, entitled “Apparatus for Energizing a Remote Station and Related Method,” U.S. Pat. No. 6,856,291, entitled “Energy Harvesting Circuits and Associated Methods,” and U.S. Pat. No. 7,057,514, entitled “Antenna on a Wireless Untethered Device such as a Chip or Printed Circuit Board for Harvesting Energy from Space,” each assigned to the assignee hereof, the disclosures of which are incorporated herein by reference.

The VNS device 125 includes an antenna 130, which, in the embodiment shown in FIG. 5, is a square spiral antenna. The antenna 130 is electrically connected to a matching network 135, which in turn is electrically connected to a voltage boosting and rectifying circuit in the form of a charge pump 140. The charge pump 140 is electrically connected to a voltage regulator 60 which is electrically connected to the control circuitry 45. The control circuitry 45 is as described above in connection with FIG. 2 and controls the generation of the current pulses provided to the probes 15 (and ultimately to the patient's vagus nerve).

In operation, the antenna 130 receives energy, such as RF energy, that is transmitted in space by a far-field RF source 145. The RF source 145 may be, without limitation, a local radio station or a dedicated base station. The RF energy received by the antenna 130 is provided, in the form of an AC signal, to the charge pump 140 through the matching network 135. The charge pump 140 amplifies and rectifies the received AC signal and provides the resulting DC signal to the voltage regulator 60. The voltage regulator 60 provides a regulated DC signal to the control circuitry 45 as a power supply. Thus, the VNS device 125 is able to be powered remotely without the need for an onboard power supply or energy storage device such as a capacitor or rechargeable battery.

The matching network 135 preferably matches the impedance of the charge pump 140 to the impedance of the antenna 130 in a manner such that the DC power output by the voltage regulator is maximized (i.e., the particular components of the matching network 135 are chosen so as to accomplish this goal). For example, the matching network 135 may be an LC tank circuit and the inductance and capacitance values thereof may be specifically chosen so as to maximize the DC power output by the voltage regulator. In one particular embodiment, the matching network is an LC tank circuit formed by the inherent distributed inductance and inherent distributed capacitance of the conducing elements of the antenna 130. Such an LC tank circuit has a non-zero resistance R which results in the retransmission of some of the incident RF energy. This retransmission of energy may cause the effective area of the antenna 130 to be greater than the physical area of the antenna 130.

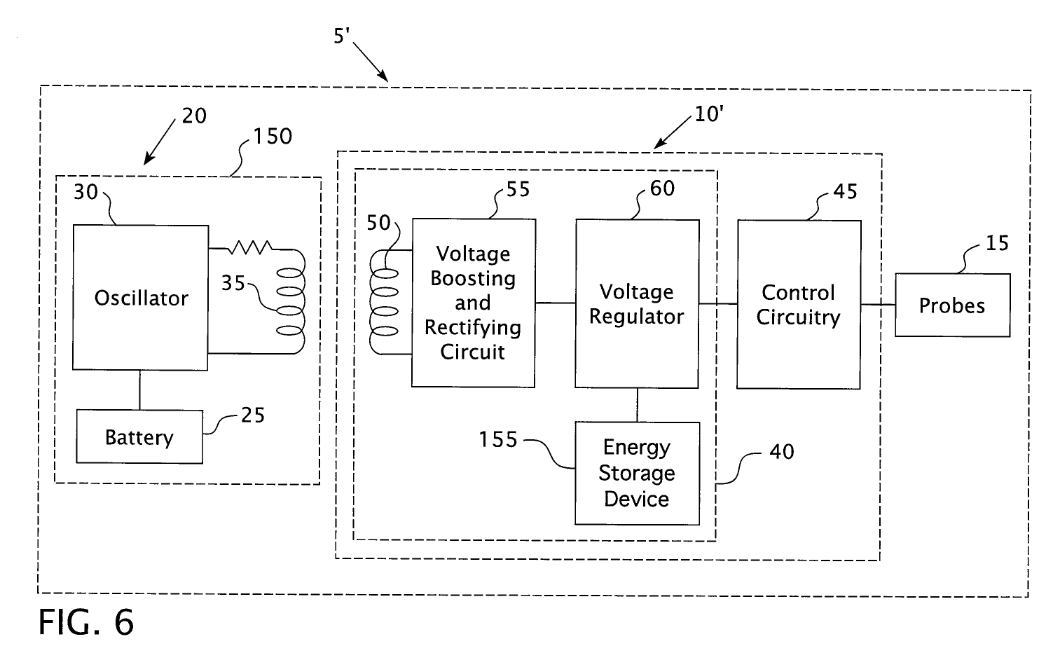

FIG. 6 is a block diagram of a VNS device 5′ according to an alternative embodiment of the present invention that is, except as described below, identical to the VNS device 5 shown in FIG. 1. In the VNS device 5′, the power supply 20 is provided in a stationary location 150, such as within the headboard of the patient's bed. The implantable device 10′ is identical to the implantable device 10 shown in FIG. 1 except that it includes power circuitry 40′ that includes an energy storage device 155, which may be, without limitation, a capacitor such as a so-called super capacitor (on the order of at least 0.2-10 F) or a rechargeable battery. In operation, the implantable device 10′ receives power from the power supply 20 by near-field inductive coupling in the manner described elsewhere herein when the implantable device 10′ is in proximity with the power supply 20. As used herein, proximity means that the secondary winding 50 is within the field generated by the primary winding 35. In the embodiment where the power supply 20 is provided in the headboard of the patient's bed, the implantable device 10′ will be in proximity with the power supply 20 when the patient is sleeping. The AC signal that is generated by the near-field inductive coupling is amplified and rectified in the manner described in connection with implantable device 10. However, in the case of the implantable device 10′, the resulting DC signal that is generated is used to: (i) power the control circuitry 45, and (ii) charge the energy storage device 155 so that the power that is stored therein may later be used to power the control circuitry 45 when the implantable device 10′ is no longer in proximity with the power supply 20. The operation of the implantable device 10′ is otherwise identical to the operation of the implantable device 10.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. An apparatus for providing electrical stimulation to the vagus nerve of a patient, comprising:
one or more probes for being implanted in the body of said patient for providing current pulses to said vagus nerve;
an implantable device for being implanted in the body of said patient, said implantable device having: (i) control circuitry electrically connected to said one or more probes, said control circuitry being structured to generate said current pulses and provide said current pulses to said one or more probes, and (ii) power circuitry electrically connected to said control circuitry for providing a DC power signal to said control circuitry; and
a power supply separate from said implantable device and external to said patient's body, said power supply providing power to said implantable device through a near-field technique between said power supply and said power circuitry when said power circuitry is in proximity with said power supply.

2. The apparatus according to claim 1, wherein said near-field technique is near-field inductive coupling between said power supply and said power circuitry.

3. The apparatus according to claim 2, wherein said power supply includes an oscillator and a primary winding, said oscillator generating a first AC signal and providing said first AC signal to said primary winding, wherein said power circuitry includes a secondary winding, wherein said first AC signal induces a second AC signal in said secondary winding when said secondary winding is in proximity with said primary winding, and wherein said power circuitry converts said second AC signal into said DC power signal.

4. The apparatus according to claim 3, wherein said power circuitry includes a voltage boosting and rectifying circuit that converts said second AC signal into a first DC signal, and a voltage regulator that receives said first DC signal and generates said DC power signal based thereon.

5. The apparatus according to claim 4, wherein said voltage boosting and rectifying circuit is a one or more stage charge pump.

6. The apparatus according to claim 1, wherein said control circuitry includes a programmable processor and a wireless communications device, said programmable processor controlling the generation of said current pulses based upon one or more pulse parameters, and wherein said apparatus further comprises a remote programming device external to said patient's body, said remote programming device being structured to wirelessly transmit programming signals to said wireless communications device, said programming signals being provided to said programmable processor for adjusting said one or more pulse parameters.

7. The apparatus according to claim 6, wherein said one or more pulse parameters specify one or more of a frequency, an amplitude, a pulse width, an on/off state, and an application location of said current pulses, said application location being determined by the particular ones of said one or more probes to which said current pulses are provided.

8. The apparatus according to claim 1, wherein said power supply is provided as part of an article to be worn by said patient.

9. The apparatus according to claim 8, wherein said article comprises a garment.

10. A method of providing electrical stimulation to the vagus nerve of a patient, comprising:
implanting one or more probes into the body of said patient, said one or more probes being structured to provide current pulses to said vagus nerve;
implanting a device in said body of said patient, said device being electrically connected to said one or more probes;
causing said device to generate said current pulses and provide said current pulses to said one or more probes; and
providing power to said device from a location external to the body of said patient using a near-field technique.

11. The method according to claim 10, wherein said near-field technique is near-field inductive coupling.

12. The method according to claim 11, wherein said step of providing power includes generating a first AC signal at said location external to the body of said patient, said first AC signal inducing a second AC signal in said device, and converting said second AC signal to a DC power signal for powering said device.

13. The method according to claim 10, wherein said current pulses are generated based upon one or more pulse parameters, the method further comprising selectively wirelessly adjusting said one or more pulse parameters from a site external to the body of said patient.

14. The method according to claim 13, wherein said one or more pulse parameters specify one or more of a frequency, an amplitude, a pulse width, an on/off state, and an application location of said current pulses.

15. An apparatus for providing electrical stimulation to the vagus nerve of a patient, comprising:
one or more probes for being implanted in the body of said patient for providing current pulses to said vagus nerve; and
an implantable device for being implanted in the body of said patient, said implantable device including:
control circuitry electrically connected to said one or more probes, said control circuitry being structured to generate said current pulses and provide said current pulses to said one or more probes, and
power circuitry electrically connected to said control circuitry, said power circuitry having an antenna for receiving energy transmitted in space from a far-field source, said power circuitry converting said received energy into a DC power signal and providing said DC power signal to said control circuitry.

16. The apparatus according to claim 15, wherein said energy transmitted in space comprises RF energy transmitted by a remote RF source.

17. The apparatus according to claim 16, wherein said remote RF source is a radio station.

18. The apparatus according to claim 15, wherein said antenna has an effective area greater than its physical area.

19. The apparatus according to claim 18, wherein said power circuitry further includes a matching network electrically connected to said antenna and a voltage boosting and rectifying circuit electrically connected to said matching network, wherein said received energy is an AC signal, and wherein said voltage boosting and rectifying circuit converts said AC signal into a DC signal.

20. The apparatus according to claim 19, wherein said matching network is an LC tank network having a non-zero resistance.

21. The apparatus according to claim 19, wherein said voltage boosting and rectifying circuit is a one or more stage charge pump.

22. The apparatus according to claim 15, wherein said implantable device does not include an energy storage device for storing power for use when said antenna is not receiving said energy transmitted in space.

23. The apparatus according to claim 15, wherein said implantable device is contained entirely within the body of said patient and does not include any physical connections external to the body of said patient.

24. The apparatus according to claim 15, wherein said control circuitry includes a programmable processor and a wireless communications device, said programmable processor controlling the generation of said current pulses based upon one or more pulse parameters, and wherein said apparatus further comprises a remote programming device external to said patient's body, said remote programming device being structured to wirelessly transmit programming signals to said wireless communications device, said programming signals being provided to said programmable processor for adjusting said one or more pulse parameters.

25. The apparatus according to claim 24, wherein said one or more pulse parameters specify one or more of a frequency, an amplitude, a pulse width, an on/off state, and an application location of said current pulses.

26. A method of providing electrical stimulation to the vagus nerve of a patient, comprising:
implanting one or more probes into the body of said patient, said one or more probes being structured to provide current pulses to said vagus nerve;
implanting a device in the body of said patient, said device being electrically connected to said one or more probes;
causing said device to generate said current pulses and provide said current pulses to said one or more probes; and
providing power to said device by receiving energy transmitted in space from a remote far-field source external to the body of said patient and converting said received energy into a DC power signal.

27. The method according to claim 26, wherein said energy transmitted in space comprises RF energy and wherein said remote source is a remote RF source.

28. The method according to claim 27, wherein said remote RF source is a radio station.

29. The method according to claim 26, wherein said current pulses are generated based upon one or more pulse parameters, the method further comprising selectively wirelessly adjusting said one or more pulse parameters from a site external to the body of said patient.

30. The method according to claim 29, wherein said one or more pulse parameters specify one or more of a frequency, an amplitude, a pulse width, an on/off state, and an application location of said current pulses.

31. A method of treating at least one of depression and epilepsy, comprising:
implanting a device in the body of a patient;
causing said device to generate and provide current pulses to the vagus nerve of said patient; and
providing power to said device from a location external to the body of said patient.

32. The method according to claim 31, wherein said step of providing power to said device from a location external to the body of said patient employs a near-field technique.

33. The method according to claim 32, wherein said near-field technique is near-field inductive coupling.

34. The method according to claim 32, wherein said step of providing power includes generating a first AC signal at said location external to the body of said patient, said first AC signal inducing a second AC signal in said device, and converting said second AC signal to a DC power signal for powering said device.

35. The method according to claim 31, wherein said current pulses are generated based upon one or more pulse parameters, the method further comprising selectively wirelessly adjusting said one or more pulse parameters from a site external to the body of said patient.

36. The method according to claim 35, wherein said one or more pulse parameters specify one or more of a frequency, an amplitude, a pulse width, an on/off state, and an application location of said current pulses.

37. The method according to claim 31, wherein said step of providing power to said device from a location external to the body of said patient includes receiving energy transmitted in space from a remote far-field source external to the body of said patient and converting said received energy into a DC power signal.

38. The method according to claim 37, wherein said energy transmitted in space comprises RF energy and wherein said remote source is a remote RF source.

39. The method according to claim 38, wherein said remote RF source is a radio station.

40. The method according to claim 31, wherein said device is implanted at a location adjacent to said vagus nerve.

41. The apparatus according to claim 1, wherein said implantable device is contained entirely within the body of said patient and does not include any physical connections external to the body of said patient.

42. The method according to claim 10, wherein said device is contained entirely within the body of said patient and does not include any physical connections external to the body of said patient.

43. The method according to claim 26, wherein said device is contained entirely within the body of said patient and does not include any physical connections external to the body of said patient.

44. The method according to claim 31, wherein said device is contained entirely within the body of said patient and does not include any physical connections external to the body of said patient.

45. The apparatus according to claim 1, wherein said power supply is provided at a stationary location separate from said implantable device and external to the body of said patient.

46. The apparatus according to claim 45, wherein said power circuitry includes an energy storage device for storing at least a portion of said power for subsequent use by said implantable device.

47. The apparatus according to claim 45, wherein said power supply is provided as part of or supported by a piece of furniture.

48. The apparatus according to claim 47, wherein said power supply is provided as part of or supported by a bed.

49. The method according to claim 10, wherein said step of providing power to said device comprises providing power to said device from a stationary location external to the body of said patient using a near-field technique.

50. The method according to claim 49, further comprising storing at least a portion of said power for subsequent use by said device.

51. The method according to claim 50, wherein the stored power is used by said device when said device is located outside of an operational range of said stationary location.

52. The method according to claim 49, wherein said stationary location comprises a piece of furniture.

53. The method according to claim 52, wherein said piece of furniture is a bed.

54. The method according to claim 31, wherein said step of providing power to said device comprises providing power to said device from a stationary location external to the body of said patient using a near-field technique.

55. The method according to claim 54, further comprising storing at least a portion of said power for subsequent use by said device.

56. The method according to claim 55, wherein the stored power is used by said device when said device is located outside of an operational range of said stationary location.

57. The method according to claim 54, wherein said stationary location comprises a piece of furniture.

58. The method according to claim 57, wherein said piece of furniture is a bed.

59. The apparatus according to claim 6, wherein said power may be provided to said implantable device and said one or more pulse parameters may be adjusted simultaneously.

60. The apparatus according to claim 24, wherein said energy may be received from said far field source and said one or more pulse parameters may be adjusted simultaneously.

61. The method according to claim 13, wherein said power providing step and said adjusting step may be performed simultaneously.

62. The method according to claim 29, wherein said power providing step and said adjusting step may be performed simultaneously.

63. The method according to claim 35, wherein said power providing step and said adjusting step may be performed simultaneously.
